(19) United States
(12) Patent Application Publication
(10) Pub. No.: US 2015/0361479 A1
(43) Pub. Date: Dec. 17, 2015

(54) IDENTIFICATION OF PRE-DIABETES USING A COMBINATION OF MEAN GLUCOSE AND 1,5-ANHYDROGLUCITOL MARKERS

(71) Applicant: GlycoMark Inc., New York, NY (US)

(72) Inventors: Eric A Button, Cary, NC (US); Robert Scott Foster, Charleston, SC (US)

(73) Assignee: GLYCOMARK, INC.

(21) Appl. No.: 14/836,436

(22) Filed: Aug. 26, 2015

Related U.S. Application Data
(62) Division of application No. 13/880,168, filed on May 29, 2013.
(60) Provisional application No. 61/394,917, filed on Oct. 20, 2010.

(51) Int. Cl. C12Q 1/54; A61B 5/00; A61B 5/151; A61B 5/145

(52) U.S. Cl. CPC C12Q 1/54; A61B 5/14532; A61B 5/7282; A61B 5/151; G01N 2400/00; G01N 2800/042

(57) ABSTRACT

Described herein is a method for determining the disease state in a patient using combined mean glucose measurements and 1,5-anhydroglucitol to identify individuals at risk for developing diabetes. The ratio of mean glucose measurements to 1,5-anhydroglucitol correlates significantly better to maximal levels of postmeal glucose levels and related measurements, than mean glucose measurements or 1,5-anhydroglucitol correlate independently.

### CROSS REFERENCE TO RELATED APPLICATIONS

This application is a division of U.S. patent application Ser. No. 13/880,168, filed on Apr. 18, 2013, which is a national phase application under 35 U.S.C. §371 of International Application Serial No. PCT/US2011/056811, filed Oct. 19, 2011, that claims benefit of priority under 35 U.S.C. §119 to U.S. Provisional Application No. 61/394,917, filed Oct. 20, 2010, the contents of each that are hereby incorporated by reference in their entirety.

### FIELD

Described herein is a method for identifying patients at risk of developing pre-diabetes, early-diabetes, diabetes, or diabetes-associated disorders such as microvascular or macrovascular disease.

### BACKGROUND

Diabetes affects over 21 million American adults, with a lifetime risk ranging from 20 to >50%, depending on sex and race. Narayan et al. (2006) Diabetes Care 29:2114-2116. Identification of diabetes, and its precursor, pre-diabetes, can permit management to prevent complications or delay progression from pre-diabetes to diabetes. Because most U.S. healthcare systems do not have systematic screening programs, many Americans with diabetes or prediabetes are often undiagnosed until clinical symptoms present. Moreover, because individuals are unaware that they have pre-diabetes, these individuals cannot initiate programs aimed at preventing progression of the disease. Cowie et al. (2009) Diabetes Care 32:287-294.

In several recent studies, it is clear that particular markers disparately identify different individuals at risk for diabetes. This is not surprising because the markers reflect different aspects of glucose metabolism. Fasting and 2-hour glucose levels reflect different pathophysiological mechanisms of abnormal glucose tolerance. The pathophysiology of isolated impaired fasting glucose (IFG) includes reduced hepatic insulin sensitivity, β-cell dysfunction, and reduced β-cell mass. Faerch et al. (2009) Diabetologia 52:1714-1723. With isolated impaired glucose tolerance (IGT), peripheral insulin sensitivity is reduced with a near-normal hepatic insulin sensitivity and progressive loss of β-cell function. In contrast with acute phase markers, the hemoglobin A1c test (A1C) is a widely used marker of chronic glycemia that reflects average blood glucose levels over 2-3 months.

A study evaluating three glycemic markers, A1C, oral glucose tolerance test (OGTT), and fasting blood glucose level (FBG), showed that a marked number of diabetes cases were preceded by elevation in only one of the markers, and with limited overlap among the three. Cederberg et al. (2010) Diabetes Care 33:2077-2083. In particular, the markers A1C, OGTT, and FBG specifically detected diabetes but were not sensitive predictors of a patient's 10-year risk of developing type-2 diabetes. The number of participants who developed diabetes with elevated A1C levels, IGT, and IFG was similar—approximately one-third; IGT had the highest prevalence in this population. Furthermore, the National Health and Nutrition Examination Surveys observed that the 2-hour glucose level is a sensitive marker for detecting impaired glucose regulation and type-2 diabetes. Cowie et al. (2009) Diabetes Care 32:287-294.

In a related study using a population subset of the National Health and Nutrition Examination Surveys, the concordance in prevalence of undiagnosed diabetes using the “new” A1C criteria (6.0 to 6.5%) was compared to criteria based on fasting plasma glucose levels and 2-hour plasma glucose levels from an oral glucose tolerance test (OGTT). Cowie et al. (2010) Diabetes Care 33:562-568. The OGTT is considered the “gold standard” for diagnosing diabetes. A1C, fasting plasma glucose levels, and 2-hour plasma glucose levels diagnosed 30%, 46%, and 90% of undiagnosed diabetes, respectively. Moreover, a relatively significant number (19%) of patients with undiagnosed diabetes were detected by fasting plasma glucose and 2-hour glucose but not by A1C.

A study recently published in the New England Journal of Medicine determined that A1C was associated with diabetes risk and more strongly associated with risks of cardiovascular disease and death from any cause as compared to fasting glucose levels. Selvin et al. (2010) New England Journal of Medicine 362:800-811. Similarly, it also was reported that A1C could be used as an alternative to fasting glucose for evaluating future diabetes risk and for detecting incident cases of diabetes. Nakagami et al. (2010) Diabetes Research and Clinical Practice 87:126-131.

A1C levels may have advantages over fasting glucose with respect to diabetes risk prediction. Fasting glucose measurements, by definition, do not reflect 2-hour postprandial glucose levels. Consequently, fasting glucose measurements alone often miss a proportion of diabetic subjects who have normal fasting glucose but elevated 2-hour postprandial glucose. On the other hand, A1C is somewhat correlated with postprandial glucose at lower ranges and correlated with fasting glucose at higher ranges. Monnier et al. (2003) Diabetes Care 26:881-885. Thus, A1C covers a wider range of diabetic pathophysiological processes than fasting glucose measurements alone. The practical advantages of A1C over fasting glucose levels (i.e., higher repeatability, no fasting requirement, and ease of use as monitoring tool), indicate that A1C is an appropriate marker for early detection of diabetes.

In summary, A1C appears to be a useful marker for predicting the risk of diabetes compared to fasting plasma glucose levels; however, A1C is less useful than measurements of 2-hour postprandial glucose concentrations in most studies.

The polyol, 1,5-anhydroglucitol (1,5-AG), is a naturally occurring monosaccharide found in food. In normoglycemic persons, plasma 1,5-AG concentrations are maintained at a steady-state level because 1,5-AG is not metabolized and is distributed throughout the body. Normally, 1,5-AG is completely reabsorbed in the proximal tubule of the kidney. However, when blood glucose concentrations reach values above the renal threshold, glucose is not completely reabsorbed by the kidney. Consequently 1,5-AG blood levels decline because of competitive inhibition of renal tubule reabsorption by the excess glucose. Previous studies have shown that hyperglycemic diabetic patients have reduced plasma concentrations of 1,5-AG; these normalize gradually in response to blood glucose lowering therapies. Thus, 1,5-AG blood levels depend on the duration and magnitude of glucosuria and on the renal threshold for glucose.

Studies have shown that 1,5-anhdyroglucitol is a robust and accurate indicator of average postprandial glucose levels over 1-2 weeks. Dungan (2008) Expert Rev. Mol. Diagn. 8:9-19. A combined measurement of mean glucose concentration (e.g., measured by A1C, fructosamine, glycated albumin, or mean glucose measurements derived from continuous glucose or fingerstick measurements) and 1,5-anhydroglucitol levels identify pre-diabetic or diabetic patients. This is because postprandial glucose measurements are more useful for predicting a risk of diabetes and associated microvascular and/or macrovascular disease than A1C or fasting glucose levels. Furthermore, mean glucose and 1,5-anhydroglucitol levels are determinable using convenient and accurate blood tests, which make these measurements amenable for large-scale screening purposes.

### SUMMARY

Described herein is the combined usage of mean glucose measurements and 1,5-anhydroglucitol levels to identify individuals with a high-risk of developing diabetes at an early stage. In particular, the ratio of mean glucose measurements to 1,5-anhydroglucitol correlates more accurately to maximal levels of postmeal glucose levels than either indicator does independently. Mean glucose measurements include mean A1C levels, fructosamine levels, glycated albumin levels, and mean glucose levels derived from glucose finger sticks or continuous glucose measurements.

Also described herein is a method for detecting a disease-state in a patient. The practitioner collects a sample of blood or biological fluid from a patient for analysis. In one aspect, the method described herein relates to a method for detecting a disease-state in a patient comprising (a) determining the mean glucose concentration; (b) determining the 1,5-anhydroglucitol concentration; and (c) calculating a ratio of the measurements of (a) to (b), wherein (a) is the antecedent (or numerator) and (b) is the consequent (or denominator). In one aspect of the method described herein, the mean glucose concentration is determined using any one of hemoglobin A1C, fructosamine, glycated albumin, fingerstick measurements, or continuous glucose monitoring. In another aspect of the method described herein, the disease-state is pre-diabetes or early-stage diabetes. In another aspect of the method described herein, the disease-state is diabetes or diabetes-associated microvascular disease. In another aspect of the method described herein, the disease-state is diabetes or diabetes-associated macrovascular disease. In another aspect of the method described herein, the ratio of mean glucose concentration to 1,5-anhydroglucitol concentration is combined with additional disease-state markers selected from the group consisting of adiponectin levels, insulin levels, or fasting glucose levels so that the identification of pre-diabetes, early-stage diabetes, diabetes, diabetes-microvascular disease, or diabetes-macrovascular disease is enhanced.

Described herein is also a method for determining the effectiveness of treatment for a disease-state comprising (a) determining the mean glucose concentration; (b) determining the 1,5-anhydroglucitol concentration; and (c) calculating a ratio of the measurements of (a) to (b), wherein (a) is the antecedent (or numerator) and (b) is the consequent (or denominator). In one aspect of the method described herein, the mean glucose concentration is determined using any one of hemoglobin A1C, fructosamine, glycated albumin, fingerstick measurements, or continuous glucose monitoring. In another aspect of the method described herein, the disease-state is pre-diabetes or early-stage diabetes. In another aspect of the method described herein, the disease-state is diabetes or diabetes-associated microvascular disease. In another aspect of the method described herein, the disease-state is diabetes or diabetes-associated macrovascular disease. In another aspect of the method described herein, the ratio of mean glucose concentration to 1,5-anhydroglucitol concentration is combined with additional disease-state markers selected from the group consisting of adiponectin levels, insulin levels, or fasting glucose levels so that the identification of pre-diabetes, early-stage diabetes, diabetes, diabetes-microvascular disease, or diabetes-macrovascular disease is enhanced.

Described herein is a kit for detecting a disease-state in a patient comprising means for (a) determining the mean glucose concentration; (b) determining the 1,5-anhydroglucitol concentration; and (c) calculating a ratio of the measurements of (a) to (b), wherein (a) is the antecedent (or numerator) and (b) is the consequent (or denominator). In one aspect of the method described herein, the mean glucose concentration is determined using any one of hemoglobin A1C, fructosamine, glycated albumin, fingerstick measurements, or continuous glucose monitoring. In another aspect of the method described herein, the kit comprising additional disease-state measurements selected from the group consisting of adiponectin levels, insulin levels, or fasting glucose levels, wherein the identification of pre-diabetes, early-stage diabetes, diabetes, diabetes-microvascular disease, or diabetes-macrovascular disease is enhanced.

### BRIEF DESCRIPTION OF THE DRAWINGS

These and other aspects of the method described herein are better understood when the following Detailed Description of the Invention is read with reference to the accompanying figures.

FIG. 1 shows a ROC Curve for 1,5-AG to detect hyperglycemic episodes for T1DM and T2DM in the full A1C range (345 hyperglycemic cases and 51 non-hyperglycemic cases). The AUC of the ROC curve is 0.79 (SE 0.038, 95% Cl=0.71-0.86, P<0.001).

FIG. 2 is a box-and-whisker plot showing summary statistics for clinical observations using the A1C/1,5-AG ratio (data are also shown in Table 6). Higher ratio values indicate a worsening diabetes disease-state. The range of the ratio is 0.20 to 2.70 with a median value of 0.53. The median value of 0.53 represents an effective cutoff point in this population. Ratio values greater than 0.53 are indicative of higher diabetes risk.

### DETAILED DESCRIPTION

Recent reports suggest that A1C, when used as the primary measurement used to reflect mean glucose levels, is a suitable screening indicator for diabetes or pre-diabetes. Compared to OGTT, A1C measurement is quicker, more convenient, and can be measured any time of day with no fasting requirement. However, in a recent study pointing out the deficiencies of A1C as a stand-alone screening test, a diagnostic cut-off point for A1C of >6.5% missed a substantial number of patients who suffered from diabetes. Fajans et al. (2009) Diabetes Suppl 1: P-2245. The majority of these patients had elevated postprandial glucose (PPG) levels.

Thus, a combination of mean glucose level and postprandial glucose level should result in a more accurate screening method for diabetes. However, while there are simple and convenient tests that provide mean glucose levels over time (i.e., A1C, fructosamine, or glycated albumin), there has not been until recently a simple and convenient test that can be used to monitor PPG levels over time. Several studies have now confirmed that the 1,5-anhydroglucitol blood test is a robust indicator of PPG levels over a period of 1-2 weeks.

A combination of mean glucose concentration and 1,5-anhydroglucitol level correlates better to maximal PPG levels (OGTT surrogate measure) than either marker individually. The combined markers serve as an accurate screening test for pre-diabetes or diabetes. The ratio of mean glucose levels to 1,5-anhydroglucitol (Mean Glucose/1,5-anhydroglucitol) is a useful diagnostic maker for the following reasons:
(1) As diabetes worsens and glucose levels increase, mean glucose levels naturally increase while 1,5-anhydroglucitol levels decrease (i.e., an inverse correlation to glucose). With mean glucose being the antecedent (or numerator) and 1,5-anhydroglucitol being the consequent (or denominator), the ratio “amplifies” the independent measurements and provides more precise discrimination of more-severe or less-severe diabetic patients.
(2) 1,5-anhydroglucitol is a measure of postprandial glucose levels above the renal threshold of glucosuria (approximately 180 mg/dL). When glucose levels are below 180 mg/dL, the 1,5-anhydroglucitol level does not accurately reflect the glucose concentration and is driven primarily by dietary factors and kidney function. Therefore, lower levels of 1,5-anhydroglucitol are better indicative of glucose levels. Because 1,5-anhydroglucitol is the consequent of the ratio, lower values (i.e., those that provide better reflection of glucose levels) are emphasized to a greater extent.
(3) In contrast to (2), at higher levels of 1,5-anhydroglucitol (e.g., where 1,5-anhydroglucitol levels are affected less by glucose levels and are affected more by dietary factors and kidney function), mean glucose level as the consequent of the ratio provides additional information on glucose levels.

### EXAMPLES

### Example 1

Correlation of Mean Glucose/1,5-AG Ratio Measures to PPG Max (OGTT Surrogate Measure)

In order to determine whether the ratio of mean glucose measurements to 1,5-anhydroglucitol (1,5-AG) correlate better with the OGTT surrogate measure or maximum postmeal glucose (PPG Max), than with either marker individually, the following ratios were correlated with PPG Max: A1C/1,5-AG, Mean Glucose (CGMS)/1,5-AG, and Fructosamine/1,5-AG. These correlations were then compared to correlations of PPG Max with each of A1C, mean glucose, and glucosamine independently. Multiple regressions were calculated for comparative purposes.

Study Summary

Patients (n=23) aged 18 to 75 with type-1 or type-2 diabetes and an A1C level between 6.5 and 8% (i.e., moderately controlled patients) with stable glycemic control were examined. A CGMS monitor was worn by the patient for two consecutive 72-hour periods and the patients also acquired 7-point fingerstick glucose profiles. Areas under the curve for glucose above 180 mg/dL (AUC₁₈₀) and mean glucose concentrations determined using CGMS over each 72-hour period were compared to the levels of 1,5-AG (μg/mL), fructosamine (μmol/L), and A1C (% Hb) at baseline (Day 1), Day 4, and Day 7. Correlation coefficients and multivariate analyses of the glucose marker relationships were examined.

Study Methodology

Patient Population

A population of 23 diabetic patients evenly distributed between patients with type-1 and type-2 disease was used in this study.

Patient Inclusion Criteria
Age 18-75, male and female;
Diagnosed with diabetes type-1 or type-2;
A1C 6.5-8 by Bayer DCA-2000 point of care meter;
Stable glycemic control as defined by no recently noted deterioration or improvement in control (patient-reported) and at least 1 prior A1C measurement in the prior 6 months with no change across measures of greater than 0.5%;
Monitoring glucose at least twice daily (for type-2 diabetes) or three or more times daily (for type-1 diabetes) by patient report.

Exclusion Criteria
Pregnancy or lactation;
Medical history of cancer, end-stage liver disease, chronic renal failure (serum creatinine >2.0 mg/dL), malnutrition (unintended weight loss >10% in one year), or connective tissue disease;
Significant anemia (hemoglobin concentration <10 g/dL), known hemoglobinopathy, recent blood donation, hemolysis, recent surgery with blood loss;
Unstable retinopathy or recent retinal procedure (<6 months ago);
Patients currently taking investigational drugs or active participants of any clinical trial;
Non-English speaking subjects;
Unwilling or unable to self-monitor blood glucose;
Hypoglycemia requiring assistance in the prior 3 months.

Sequence of Study Events

Day 1

Blood was drawn and 1,5-AG, A1C, fructosamine, and fasting plasma glucose (FPG) analyses were performed. The Continuous Glucose Monitoring System (CGMS) device was inserted and the patient was taught how to manage the device.

Day 4

Blood tests were repeated and the CGMS sensor was replaced at a new site. A 24-hour urine sample was collected on Day 3 and submitted for analysis on Day 4. Glucose logs were collected and data acquired by the meters were downloaded.

Day 7

The blood tests were repeated. The CGMS device was removed and the site was inspected. Glucose logs were collected and data from the CGMS were downloaded.

Continuous Glucose Monitoring System Device

Patients wore a subcutaneously inserted CGMS (MiniMed) device that was inserted on Day 1 and removed on Day 7. The insertion site was changed on Day 4. The device was used according to FDA-approved labeling. A trained healthcare professional introduced the sensor using local antiseptic into the skin of the abdomen using an automatic insertion device and an introducer needle that were removed immediately. The sensor lies just beneath the skin and is secured with tape. The sensor was connected to a monitor that records measurements that were accessible only after downloading to a computer at the healthcare provider's office.

Fingerstick Glucose

Patients were asked to obtain fingerstick glucose measurements and keep a log of morning fasting, pre-meal, 2-hour postprandial, and bedtime glucose levels (˜7 times) daily for Days 1-6 of the study.

Maximal Postmeal Glucose

Maximal Postmeal Glucose (PPG Max) is the maximum height of each postmeal glucose excursion. PPG Max was determined and averaged for each patient for three meals (breakfast, lunch, and dinner).

Correlations to PPG Max (OGTT Surrogate Measure)

Table 1 shows correlations of A1C, Mean Glucose, and Fructosamine levels to PPG Max, a surrogate measure of OGTT. The ratio of mean glucose measures (i.e., Variable/1,5-AG=A1C/1,5-AG, Mean Glucose (Sensor)/1,5-AG, or Fructosamine/1,5-AG) were correlated to PPG Max. Multiple regressions were calculated where the Variable (A1C, Mean Glucose (Sensor), or Fructosamine levels) and 1,5-AG were independent variables and PPG Max was the dependent variable.

TABLE 1
Correlations of PPG Max

| | | Mean Glucose | |
|---|---|---|---|
| | A1C | (Sensor) | Fructosamine |
| | R = +0.30 | R = +0.47 | R = +0.16 |
| | P = 0.16 | P = 0.02 | P = 0.46 |
| Ratio | R = +0.68 | R = +0.73 | R = +0.65 |
| Variable/1,5-AG | P = 0.0004 | P = 0.00007 | P = 0.0008 |
| Regression | R = +0.50 | R = +0.66 | R = +0.55 |
| Variable and 1,5-AG | P = 0.06 | P = 0.003 | P = 0.06 |

R = Pearson Correlation Coefficient;
P = P-value;
all correlations correlate to OGTT surrogate-PPG Max
Variable = A1C, Mean Glucose (Sensor), or Fructosamine;
correlation of 1,5-AG to PPG Max-R = −0.50.

The ratio of mean glucose measures (i.e., A1C/1,5-AG, Mean Glucose (Sensor)/1,5-AG, and Fructosamine/1,5-AG) correlated significantly better to PPG Max then A1C, Mean Glucose, or Fructosamine alone—with P-values decreasing quite dramatically with use of the ratio for any mean glucose measure.

These data indicate that the combination of mean glucose measures and 1,5-AG in the form of a ratio where the mean glucose measures (A1C, Mean Glucose (Sensor), or Fructosamine) is the antecedent (or numerator) and 1,5-anhydroglucitol is the consequent (or denominator) correlates significantly better to PPG Max than the mean glucose measures or 1,5-AG levels correlate individually.

Furthermore, when combining the mean glucose measures and 1,5-AG levels in a multiple regression where PPG Max is the dependent variable, the correlation coefficients increase relative to correlations of the mean glucose variables alone to PPG Max. However, the ratio of mean glucose measures to 1,5-AG correlates better to PPG Max than the multiple regressions. Therefore, the mathematical ratio of mean glucose measures to 1,5-AG provides more accurate correlations to PPG Max than a simple combination of these variables in multiple regressions.

Collectively, these data indicate that a combination of mean glucose measurements and 1,5-anhydroglucitol in the form of a ratio correlate better to maximal PPG levels (OGTT surrogate measure) than either marker does individually.

### Example 2

Multiple Regressions of Glycemic Variables and Ratio to PPG Max (OGTT Surrogate Measure) Clinical Study Design

The design of the clinical investigation was carried out as in Example 1. In order to determine the strength of the ratio of mean glucose measures to 1,5-AG, the A1C/1,5-AG ratio, A1C, 1,5-AG, Fructosamine, and Fasting Glucose levels were incorporated into a multiple regression as independent variables. PPG Max was the dependent variable. Results are shown in Table 2.

TABLE 2
Multiple Regression Equations

| Independent Variables | Coefficient | Std. Error | t | P |
|---|---|---|---|---|
| (Constant) | 182.58 | | | |
| A1C/1,5-AG Ratio | 37.19 | 14.03 | 2.65 | 0.02 |
| A1C | 2.67 | 28.06 | 0.10 | 0.93 |
| 1,5-AG | −0.20 | 2.80 | −0.07 | 0.94 |
| Fasting Glucose | −0.09 | 0.23 | −0.37 | 0.71 |
| Fructosamine | −0.10 | 0.21 | −0.46 | 0.65 |

Correlation Coefficient: R = 0.69.

In the above regression analyses, the only independent variable that was significantly correlated to PPG Max was the ratio of A1C/1,5-AG. In other words, there is no convincing evidence that any of the other independent variables add to the predictability of PPG Max once the ratio of A1C/AG is known. These results provide additional evidence of the precision of the ratio of mean glucose measure to 1,5-AG.

### Example 3

Correlation of A1C/1,5-AG Ratio to Related Measures of PPG Max (OGTT Surrogate)

In order to validate results obtained in Examples 1 and 2, the A1C/1,5-AG ratio (as an example of a Mean Glucose/1,5-AG ratio) was correlated to related measures of PPG Max (i.e., OGTT Surrogate Measure). Measures related to PPG Max include overall hyperglycemia (AUC₁₈₀) and glycemic variability (SD, MAGE, CONGA).

Study Methodology

Between January 2006 and March 2008, study subjects were recruited at 11 international centers. Participants between 18 and 70 years of age were selected based upon stable glycemic control as evidenced by two A1C values within one percentage point of each other in the six months prior to recruitment. Individuals with a wide range of A1C levels were included. The non-diabetic (non-DM) controls had plasma glucose levels <5.4 mmol/L (97 mg/dL) after overnight fasting, A1C levels <6.5%, and no history of diabetes. Individuals with conditions that could result in major changes in glycemia (e.g., disease or pregnancy), interfere with the A1C assays (e.g. haemoglobinopathies), or with a relationship between A1C and plasma glucose concentrations (e.g. anemia, severe renal or liver disease) were excluded from the study. Because this study was observational, diabetes management was left to the patients and their usual health care providers. Further clinical data collected at the study baseline included anthropometric measurements and self-reported data on treatment.

Between April 2006 and August 2007, subjects were recruited from 10 clinical centers: 6 in the U.S., 3 in Europe, and 1 in Cameroon. Baseline measurements were completed with 708 subjects, 343 T1DM (47, 5%), 264 T2DM (36, 6%) patients and 101 non-DM controls (15, 9%). After excluding the subjects who did not have acceptable samples for A1C measurement or 1,5-AG levels measured, those who did not have adequate CGM and in whom calculation of mean blood glucose, AUC₁₈₀, or glycemic variability measures was not possible, 396 diabetic subjects and 61 non-diabetic controls remained.

Measures of Glycemia (A1C, 1,5-AG, CGM)

A1C samples from baseline visit were analyzed in a central laboratory with four different DCCT-assays that were aligned with the National Glycohemoglobin Study Program: 1,5-AG from baseline visit was measured on frozen samples centrally in a local laboratory by an automated enzymatic colorimetric assay for 1,5-AG (GlycoMark; Winston-Salem, NC). Measures of glycemia included continuous interstitial glucose monitoring (CGM; Medtronic Minimed, Northridge, CA) that was performed for at least two days at baseline and at the end of each month for three months. For calibration purposes and as an independent measure of glycemia, subjects were asked to perform 8-point (pre-meals, 90 minutes post-meals, pre-bed time and at 3 AM) self-monitoring of capillary glucose with the HemoCue blood glucose meter (HemoCue Glucose 201 plus, HemoCue, Angelholm, Sweden) during the two days of CGM. The data were downloaded and exported to the data-coordinating center. To be acceptable for analysis, CGM data had to include at least one successful 24-hour profile out of the two to three days of monitoring with no gaps >120 minutes, and a mean absolute difference compared with the HemoCue calibration results <18%, as recommended by the manufacturer.

Measures of Glycemic Variability

Measurements of average glucose level, glycemic variability, and hyperglycemic episodes were based on CGM data from a 48-hour monitoring period at the baseline visit and were calculated after exclusion of the initial 2 hours of monitoring, which is considered an unstable calibration period (see Table 3). Three indices of glycemic variability were calculated based on CGM: the standard deviation (SD) of all glucose values, the Mean Amplitude of Glycemic Excursions (MAGE) and the Continuous Overlapping Net Glycemic Action (CONGA).

MAGE is the mean of the differences between consecutive peaks and nadirs, only including changes of more than 1 SD of glycemic values, thus capturing only major fluctuations. It has been shown to be independent of mean glycemia. For the calculation of CONGA, n=4, the difference of the current observation and the observation 4 hours previously is calculated for each observation after the first 4 hours. The CONGA 4 is the SD of these differences and measures the overall intra-day variation of glucose recordings during 4-hour periods.

Higher SD, MAGE, and CONGA values indicate greater glycemic variability. The area under the glucose curve was determined above the 180 mg/dL (AUC₁₈₀) level using CGM data. This was used as a measure of general hyperglycemia above the renal threshold of glucose. Also from CGM, a postprandial AUC (AUCpp) was calculated for periods of 2 or 4 hours after a meal. This was only possible in a limited number of patients.

Statistical Analyses

Bivariate associations (Pearson partial correlations) between 1,5-AG (log transformed) and MBG (mean blood glucose), SD, MAGE, CONGA, AUC₁₈₀, and AUCpp obtained from CGM data (from a limited patient group). The ratio of A1C/1,5-AG was correlated to these parameters. Calculations were performed with patients representing the entire range of A1C levels (n=396) and for patients with A1C levels below or equal to 8.0% (n=290). Data are shown in Table 3.

TABLE 3
Measures of Glycemic Variability

| Correlation | A1C/1,5-AG | 1,5-AG | A1C/1,5-AG | |
|---|---|---|---|---|
| Coefficients | Ratio | Full A1C | Ratio | 1,5-AG |
| for | Full A1C Range | range | A1C ≦8% | A1C ≦8% |
| N = | 396 | 396 | 290 | 290 |
| MBG | 0.605** | −0.530** | 0.453** | −0.398** |
| SD | 0.479** | −0.440** | 0.467** | −0.429** |
| MAGE | 0.363** | −0.337** | 0.366** | −0.333** |
| CONGA4 | 0.445** | −0.414** | 0.440** | −0.401** |
| AUC₁₈₀ | 0.492** | −0.430** | 0.372** | −0.339** |
| N = | 210 | 210 | 153 | 153 |
| AUCpp 2 hours | 0.460** | −0.416** | 0.321** | −0.304** |
| AUCpp 4 hours | 0.458** | −0.413** | 0.321** | −0.301** |

Correlation Coefficients of bivariate associations (partial correlations) of log (A1C/1,5-AG) ratio, 1,5-AG (log transformed), stratified for A1C, and measures of glycemic control and GV, postprandial and overall hyperglycemia in T1DM and T2DM pooled, adjusted for diabetes type, sex and age. The AUCpp-values were not available in all patients but only in a smaller sample size; correlation is significant;
P-value <0.05* or <0.01**

All correlations of 1,5-AG independently and the A1C/1,5-AG ratio to glycemic measures were statistically significant (all P-values <0.01). In all cases, the A1C/1,5-AG ratio correlated better to AUC and glycemic variability measures than 1,5-AG alone. This comparative correlation was more apparent in patients with A1C levels less than 8.0%, including patients with A1C levels in the normal range. These data show that the A1C/1,5-AG ratio provides more accurate correlations to related measures of PPG Max (i.e., AUC₁₈₀, SD, MAGE, or CONGA). See supporting data in Examples 1 and 2.

### Example 4

A1C Alone is Not Sufficient to Detect Hyperglycemic Excursions/Early-Stage Diabetes (1,5-AG is Necessary)

Receiver Operating Characteristic (ROC) analyses were performed to examine the test performance of 1,5-AG in detecting hyperglycemic episodes using data obtained in the study described in Example 3. Because 1,5-AG is cleared renally by competitive inhibition above a renal threshold of approximately 180 mg/dL, the test performance of 1,5-AG was defined to detect hyperglycemic episodes as defined by AUC₁₈₀ mg/dL. This was analyzed at different levels of A1C. This test determines whether 1,5-AG's performance is truly significant compared to the null hypothesis (true area=0.5) or is it only better by chance. The 95% CI and P-values are asymptotic.

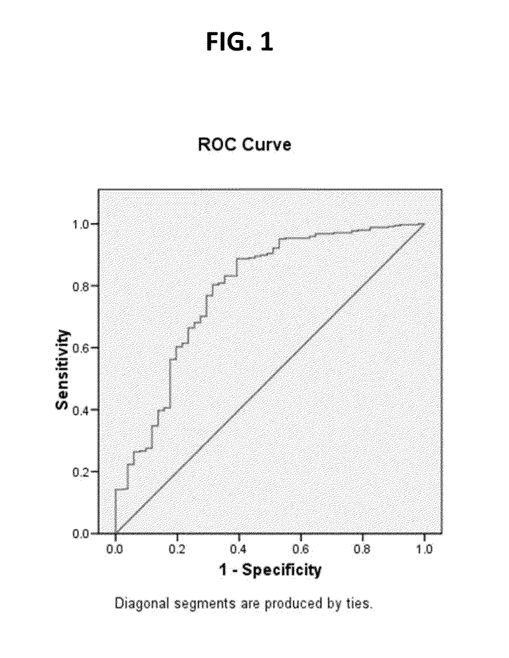

The ROC analysis was performed on only the patients with DM in the full A1C range (345 hyperglycemic and 51 non-hyperglycemic). The area under the ROC curve was 0.79 (SE 0.038, 95% Cl=0.71-0.86, P<0.001) (see FIG. 1). This value ranged from 0.68 (SE 0.079 95% CI =0.34-0.535, P<0.08) in the A1C group 6% (21 hyperglycemic and 28 non-hyperglycemic) to 0.73 (SE 0.046, 95% Cl=0.64-0.82, P<0.001) in the A1C group 8% (240 hyperglycemic and 50 non-hyperglycemic).

These result show that a significant number of patients with good to moderate glycemic control experienced hyperglycemic episodes, and even at A1C values <6.0%, 21 out of 49 patients (43%) were hyperglycemic. In other words, A1C measurements alone miss glycemic excursions in patients who would have been classified as “normal.” As seen in the ROC analysis, 1,5-AG readily detects hyperglycemic excursions, even in the A1C normal range. These results underscore the need for a combination of A1C and 1,5-AG to detect early stage diabetes. As described in the other Examples, the ratio of A1C (and other mean glucose measures) to 1,5-AG is an effective mathematical combination.

### Example 5

Clinical Utility of Mean Glucose Measures/1,5-AG Ratio

In order to show the practical clinical utility of using the ratio of mean glucose to 1,5-AG, 21 patients in the normal/pre-diabetic A1C range was analyzed. The levels of A1C, 1,5-AG, and the ratio of A1C/1,5-AG values for these patients are shown in Table 4.

TABLE 4
Clinical Utility of Mean Glucose Measures/1,5-AG Ratio

| A1C (%) | 1,5-AG (μg/mL) | A1C/1,5-AG Ratio |
|---|---|---|
| 5.0 | 9.5 | 0.53 |
| 5.2 | 26.0 | 0.20 |
| 5.3 | 7.1 | 0.75 |
| 5.3 | 16.8 | 0.32 |
| 5.5 | 16.5 | 0.33 |
| 5.5 | 5.7 | 0.96 |
| 5.6 | 21.0 | 0.26 |
| 5.7 | 11.4 | 0.50 |
| 5.7 | 23.2 | 0.25 |
| 5.7 | 4.9 | 1.16 |
| 5.8 | 11.8 | 0.49 |
| 5.9 | 6.9 | 0.85 |
| 5.9 | 6.6 | 0.89 |
| 6.0 | 14.1 | 0.43 |
| 6.0 | 18.4 | 0.32 |
| 6.3 | 10.2 | 0.62 |
| 6.3 | 9.2 | 0.68 |
| 6.3 | 4.3 | 1.46 |
| 6.4 | 2.3 | 2.70 |
| 6.4 | 16.8 | 0.38 |
| 6.4 | 6.3 | 1.02 |

In Table 5, patients were grouped by 1,5-AG levels greater than and less than 12 μg/mL. At 1,5-AG levels less than 12 μg/mL, 1,5 AG detects glucose excursions greater than 180 mg/dL. Mean A1C levels and the mean of the A1C/1,5-AG ratio were calculated for each population. T-tests (independent samples) were performed to determine whether there were significant differences.

TABLE 5
Comparison of Patients with Higher and Lower Glycemic Excursions

| Patient Group | Mean A1C (%) | Mean A1C/1,5-AG Ratio |
|---|---|---|
| 1,5-AG <12 μg/mL (n = 13) | 5.71 | 0.31 |
| 1,5-AG >12 μg/mL (n = 8) | 5.89 | 0.97 |
| | P = 0.39 | P = 0.006 |

There was no significant difference between mean A1C levels in the populations (P-value =0.39), meaning that A1C levels alone could not differentiate/detect glycemic excursions—which is essential for identifying early-stage diabetes. However, the ratio of A1C/1,5-AG readily differentiates these patients (P-value=0.006), thus underscoring the power of the ratio to identify patients with early stage diabetes.

A1C/1,5-AG Ratio—Summary Statistics (21 patients from Clinic)

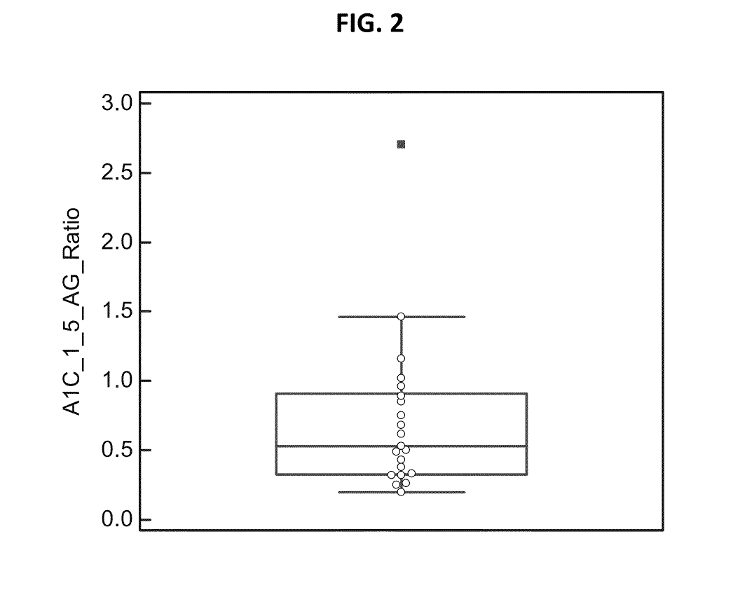

Summary statistics for the A1C/1,5-AG ratio are shown in Table 6 and in FIG. 2. Higher ratio values indicate a worsening diabetes state. The range of the ratio is 0.20 to 2.70 with a median value of 0.53. The median value of 0.53 represents an effective cutoff point in this population. Ratio values greater than 0.53 are indicative of higher diabetes risk. 10 patients had ratio values greater than 0.53, with 5 of these patients having A1C values in the normal range. These patients would have been classified as being “normal” by A1C values even though these patients are pre-diabetic.

TABLE 6
Summary Statistics for the A1C/1,5-AG Ratio

| Variable | A1C/AG Ratio |
|---|---|
| Sample size | 21 |
| Lowest value | 0.2000 |
| Highest value | 2.7000 |
| Arithmetic mean | 0.7190 |
| 95% CI for the mean | 0.4620 to 0.9761 |
| Median | 0.5300 |
| 95% CI for the median | 0.3580 to 0.8676 |
| Variance | 0.3190 |
| Standard deviation | 0.5648 |
| Relative standard deviation | 0.7855 (78.55%) |
| Standard error of the mean | 0.1232 |
| Coefficient of Skewness | 2.3606 (P = 0.0001) |
| Coefficient of Kurtosis | 7.1237 (P = 0.0008) |
| D'Agostino-Pearson test for | reject Normality (P < 0.0001) |
| Normal distribution | |

| Percentiles | | 95% Confidence Intervals |
|---|---|---|
| 2.5 | 0.2012 | |
| 5 | 0.2275 | |
| 10 | 0.2560 | |
| 25 | 0.3275 | 0.2517 to 0.4982 |
| 75 | 0.9075 | 0.6308 to 1.4085 |
| 90 | 1.2800 | |
| 95 | 2.0180 | |
| 97.5 | 2.6690 | |

### Example 6

Using the Mean Glucose/1,5-AG Ratio to Predict Diabetes and Diabetes-Associated Microvascular and Macrovascular Disease

A study is examining Mean Glucose/1,5-AG ratio measurements in 14,166 existing stored specimens from participants from the ARIC Study (see additional study details below). The following ratios are being tested: A1C/1,5-AG, Fructosamine/1,5-AG, Glycated Albumin/1,5-AG, and other mean glucose measures/1,5-AG.

This study characterizes the epidemiologic associations and evaluates the contributions of Mean Glucose/1,5-AG ratio measurements to predict the incidence of diabetes, microvascular disease (i.e., kidney disease and retinopathy), and macrovascular disease in a community-based population. It is thought that Mean Glucose/1,5-AG ratio measurements provide better prognostic information than known glycemic markers alone (fasting glucose and A1C) for predicting the outcomes of microvascular and macrovascular diseases.

This study also compares and contrasts racial differences in absolute levels of Mean Glucose/1,5-AG ratio measurements. In addition differences in prediction of clinical outcomes (retinopathy, kidney disease, cardiovascular disease, and all-cause mortality) in persons with and without diabetes.

Racial differences in Mean Glucose/1,5-AG ratio measurements can provide independent confirmation of real racial disparities in glycemia (as opposed to mere racial differences in the tendency for hemoglobin to become glycosylated). Differences in glucose homeostasis preceding the development of diabetes and suboptimal glycemic control in the setting of diabetes should partly explain racial differences in risk of diabetes and diabetic complications, particularly microvascular disease.

This study also characterizes the association of Mean Glucose/1,5-AG ratio measurements and its trajectory across the life-course—from mid-life to older age—with measures of frailty, mood, and physical and cognitive function in elderly adults. Current post-prandial hyperglycemia and historical trajectories in post-prandial hyperglycemia, as measured by Mean Glucose/1,5-AG ratio measurements, contribute to frailty, dementia, poor mood, and cognitive and physical impairment in elderly adults.

Mean Glucose/1,5-AG ratio measurements can provide additional prognostic information for the prediction (risk) of diabetes, and microvascular/macrovascular outcomes. Microvascular outcomes include but are not limited to retinopathy and kidney disease. Macrovascular outcomes include but are not limited to coronary heart disease, ischemic stroke, and death from any cause.

The Atherosclerosis Risk in Communities (ARIC) Study Background

GMAS is an Approved Ancillary Study that will be nested within the ongoing Atherosclerosis Risk in Communities (ARIC) Study. The ARIC Study is an on-going NHLBI-funded community-based longitudinal cohort study of 15,792 black and white adults aged 45-64 years at baseline sampled from 4 U.S. communities. The ARIC Study is one of the most important long-term studies of subclinical and clinical atherosclerotic disease in the U.S. The first clinic examinations (Visit 1) took place during 1987-1989, with three follow-up visits approximately every three years. A wealth of information on cardiovascular and diabetes risk factors, including lipids, anthropometric data, systolic and diastolic blood pressures, socio-demographic, behavioral, dietary intake, and lifestyle information is available for all participants. Ascertainment of cardiovascular events in the ARIC cohort is comprehensive and utilizes multiple data sources to confirm cases. Extensive information is also available on kidney disease and retinopathy (retinal photography) in all participants, at multiple time points during follow-up. All living ARIC Participants (8,000) will be invited back for a planned Visit 5 to be conducted in the years 2011-2013, during which an extensive medical examination will take place including blood and urine sample collection.

What is claimed is:

1. A kit for detecting a disease-state in a patient comprising means for:
(a) determining the mean glucose concentration;
(b) determining the 1,5-anhydroglucitol concentration; and
(c) calculating a ratio of the measurements of (a) to (b),
wherein the mean glucose concentration is determined using any one of hemoglobin A1C, fructosamine, glycated albumin, fingerstick measurements, or continuous glucose monitoring; and
wherein the disease-state is pre-diabetes, early-stage diabetes, diabetes, diabetes-associated microvascular disease, or diabetes-associated macrovascular disease.

2. The kit of claim 1, comprising additional disease-state measurements selected from the group consisting of adiponectin levels, insulin levels, or fasting glucose levels,
wherein the identification of pre-diabetes, early-stage diabetes, diabetes, diabetes-microvascular disease, or diabetes-macrovascular disease is enhanced.

3. The kit of claim 1, wherein the mean glucose concentration is determined using any one of hemoglobin A1C, fructosamine, glycated albumin, fingerstick measurements, or continuous glucose monitoring.

4. The kit of claim 1 or 2, wherein the disease-state is pre-diabetes or early-stage diabetes.

5. The kit of claim 1 or 2, wherein the disease-state is diabetes or diabetes-associated microvascular disease.

6. The kit of claim 1 or 2, wherein the disease-state is diabetes or diabetes-associated macrovascular disease.

7. The kit of claim 1 or 2, wherein the ratio of mean glucose concentration to 1,5-anhydroglucitol concentration is combined with additional disease-state markers selected from the group consisting of adiponectin levels, insulin levels, or fasting glucose levels,
wherein the identification of pre-diabetes, early-stage diabetes, diabetes, diabetes-microvascular disease, or diabetes-macrovascular disease is enhanced.

8. A kit for detecting a disease-state in a patient comprising:
(a) determining the mean glucose concentration;
(b) determining the 1,5-anhydroglucitol concentration; and
(c) calculating a ratio of the measurements of (a) to (b),
wherein the mean glucose concentration is determined using any one of hemoglobin A1C, fructosamine, glycated albumin, fingerstick measurements, or continuous glucose monitoring; and
wherein the disease-state is pre-diabetes, early-stage diabetes, diabetes, diabetes-associated microvascular disease, or diabetes-associated macrovascular disease.

9. The kit of claim 8, wherein the ratio of mean glucose concentration to 1,5-anhydroglucitol concentration is combined with additional disease-state markers selected from the group consisting of adiponectin levels, insulin levels, or fasting glucose levels,
wherein the identification of pre-diabetes, early-stage diabetes, diabetes, diabetes-microvascular disease, or diabetes-macrovascular disease is enhanced.

10. A kit for determining the effectiveness of treatment for a disease-state comprising:
(a) determining the mean glucose concentration;
(b) determining the 1,5-anhydroglucitol concentration; and
(c) calculating a ratio of the measurements of (a) to (b),
wherein the mean glucose concentration is determined using any one of hemoglobin A1C, fructosamine, glycated albumin, fingerstick measurements, or continuous glucose monitoring; and
wherein the disease-state is pre-diabetes, early-stage diabetes, diabetes, diabetes-associated microvascular disease or diabetes-associated macrovascular disease.

11. The kit of claim 10, wherein the ratio of mean glucose concentration to 1,5-anhydroglucitol concentration is combined with additional disease-state markers selected from the group consisting of adiponectin levels, insulin levels, or fasting glucose levels,
wherein the identification of pre-diabetes, early-stage diabetes, diabetes, diabetes-microvascular disease, or diabetes-macrovascular disease is enhanced.
